(19) United States
(12) Patent Application Publication
(10) Pub. No.: US 2006/0217717 A1
(43) Pub. Date: Sep. 28, 2006

(54) Methods and devices for stabilizing a bone anchor

(76) Inventor: Dale Whipple, East Taunton, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/088,345

(22) Filed: Mar. 24, 2005

(51) Int. Cl. A61F 2/30

(52) U.S. Cl. 606/61

(57) ABSTRACT

A stabilizer for a bone anchor includes a tubular body having an outer wall and an inner wall and a plurality of projections spaced about and extending from the outer wall of the tubular body. The inner wall defines a lumen sized and shaped to receive a portion of the bone anchor and the projections are configured to engage bone to inhibit rotation of the stabilizer and distribute forces on the bone anchor to the bone.

### BACKGROUND

Bone anchors, such a polyaxial and monoaxial bone screws, are used in treatment of spinal disorders, such as spinal deformities and degeneration, to facilitate realignment and/or stabilization of the vertebrae of the spine. Once implanted, a significant amount of the force applied to the bone anchor is perpendicular to the axis of the bone anchor. Such forces may be concentrated on the cortical bone adjacent the bone anchor, which can cause the cortical bone to weaken, particularly if the cortical bone is osteoporotic. As a result of the weakened bone, the bone anchor may loosen from the bone and, in some cases, may back out.

### SUMMARY

Disclosed herein are methods and devices for stabilizing a bone anchor, such as a polyaxial or monoaxial bone screw used to treat spinal disorders. The stabilizing devices disclosed herein provide increased surface area for contact with bone and, thus, operate to distribute the lateral forces applied to the bone anchor over an increased area of bone.

In accordance with one exemplary embodiment, a stabilizer for a bone anchor may comprise a tubular body having an outer wall and an inner wall and a plurality of projections spaced about and extending from the outer wall of the tubular body. In the exemplary embodiment, the inner wall defines a lumen sized and shaped to receive a portion of the bone anchor and the projections are configured to engage bone and distribute forces on the bone anchor to the bone.

In accordance with another exemplary embodiment, a spinal bone anchor assembly may comprise a bone screw having a proximal head and a distal shaft, a receiver member having a proximal portion for receiving a spinal fixation element and a distal portion for receiving the proximal head of the bone screw, and a stabilizer having a tubular body positioned about a portion of the shaft of the bone screw and a plurality of projections spaced about and extending from the tubular body. In the exemplary embodiment, the projections are configured to engage bone to inhibit rotation of the stabilizer and distribute forces on the bone anchor to the bone.

In accordance with another exemplary embodiment, a method of inserting a bone screw into a vertebra may comprise creating a hole in a portion of the vertebra, inserting a stabilizer in the hole, the stabilizer having a tubular body and a plurality of projections spaced about and extending from the tubular body, and positioning the shaft of a bone screw in the stabilizer.

### BRIEF DESCRIPTION OF THE FIGURES

These and other features and advantages of the devices and methods disclosed herein will be more fully understood by reference to the following detailed description in conjunction with the attached drawings in which like reference numerals refer to like elements through the different views. The drawings illustrate principles of the devices and methods disclosed herein and, although not to scale, show relative dimensions.

FIG. 1 is a perspective view of an exemplary embodiment of a stabilizer for a bone anchor;

FIG. 2 is a side elevational view in cross section of the stabilizer of FIG. 1;

FIG. 3 is a perspective view of the stabilizer of FIG. 1 coupled to a bone anchor assembly;

FIG. 4 is a side elevational view in cross section of the stabilizer and bone anchor assembly of FIG. 3, illustrating the stabilizer and bone anchor assembly implanted in a vertebra; and

FIG. 5 is a perspective view of view of the stabilizer of FIG. 1 and insertion instrument for positioning the stabilizer.

### DETAIL DESCRIPTION OF EXEMPLARY EMBODIMENTS

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The articles “a” and “an” are used herein to refer to one or to more than one (i.e. to at least one) of the grammatical object of the article. By way of example, “an element” means one element or more than one element.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

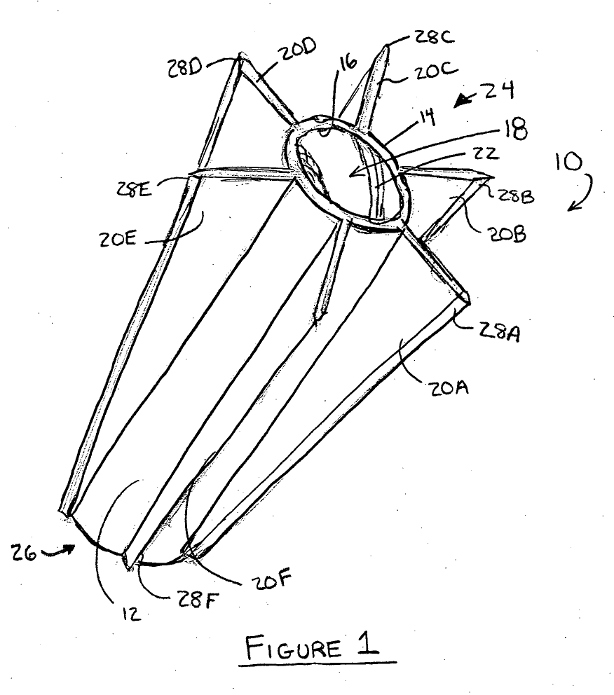

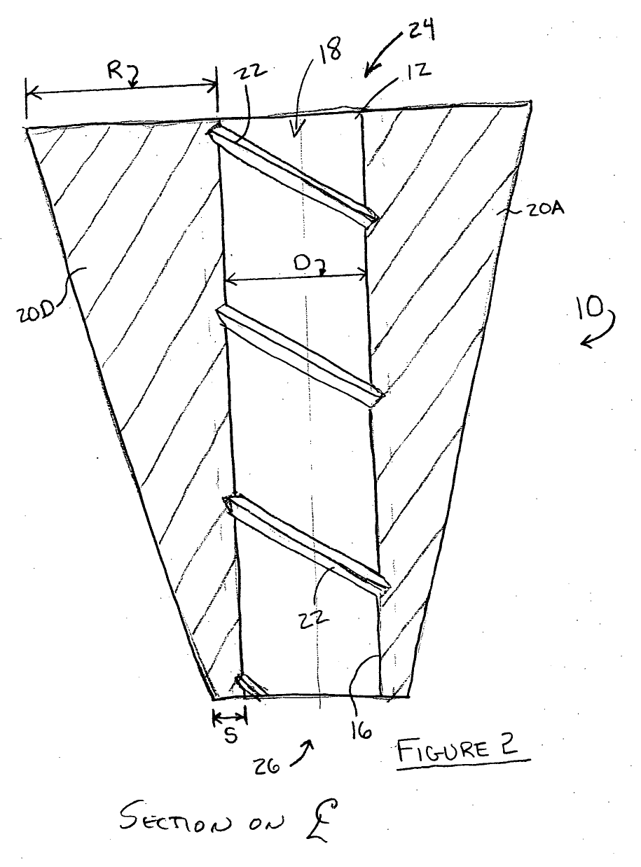

FIGS. 1-2 illustrate an exemplary embodiment of a stabilizer 10 for a bone anchor. The exemplary stabilizer 10 includes a tubular body 12 having an outer wall 14 spaced apart from an inner wall 16. The exemplary stabilizer 10 also includes a plurality of projections 20 spaced about and extending from the outer wall 14 of the tubular body 12. The projections 20, in the exemplary embodiment, may be configured to engage bone to stabilize a bone anchor, such as polyaxial or monoaxial bone screw, by, for example, inhibiting rotation of the stabilizer and bone anchor relative to the bone and distributing forces on the bone anchor to the bone.

In the exemplary embodiment, the body 12 is approximately cylindrical in shape having an approximately circular cross section. One skilled in the art will appreciate that the body 12 may have other cross sectional shapes, including elliptical, rectilinear, triangular, or polygonal. The inner wall 16 of the body 12 defines a lumen 18 that is sized and shaped to receive a portion of a bone anchor, such as the threaded shaft of a bone screw. For example, in certain exemplary embodiments, the extent, e.g., the diameter D, of the inner wall 16 of the body 12 may be approximately equal to the major diameter of the threaded shaft of the bone screw received in the lumen 18. In certain alternative embodiments, the extent, e.g., the diameter D of the inner wall 16 may be greater that or approximately equal to a non-threaded proximal portion of the shaft of the bone screw. In certain embodiments, the inner wall 16 of the body 12 may include internal threads 22 for engaging the bone engaging threads of the shaft of a bone screw as the bone screw is positioned relative to the stabilizer 10. The internal threads 22 may be complementary to the external bone engaging threads on the shaft of the bone screw. In certain alternative embodiments, the inner wall 16 may lack internal threads and may have a smooth bore.

The exemplary stabilizer 10 may include any number of projections 20, e.g., one or more projections. For example, the exemplary stabilizer 10 includes six projections 20A-F spaced about the circumference of the outer wall 14 of the tubular body 12. The projections 20A-F, in the exemplary embodiment, are generally planar in shaped and extend radially from the outer wall 14. The projections 20 may taper from a proximal end 24 of the tubular body 12 to a distal end 26 of the tubular body 12 to facilitate insertion of the stabilizer 10 into bone. For example, in the exemplary embodiment, the projections 20A-F extend radially a distance R at the proximal end 24 of the tubular body 12 and extend radialy a distance S, which is less than distance R, at the distal end 26 of the tubular body 12. For a bone screw used in posterior lumbar spine surgery R may be approximately 10 mm to approximately 1 mm and S may be approximately 2 mm to approximately 0 mm.

In the exemplary embodiment, the projections 20A-F taper linearly from the proximal end to the distal end of the tubular body 12. In alternative embodiments, the projections 20 may taper non-linearly from the proximal end 24 to the distal end 26 of the tubular body 12.

Each projection 20 may be similarly sized and shaped, as in the illustrated embodiment, or one or more of the projections may have a different size and/or shape than other projections. The projections 20 may be spaced equally about the outer wall 14 of the tubular body 12, as in the illustrated embodiment, or one or more projections may be spaced a distance apart from other projections that is different the distance between other projections.

One or more of the projections 20 of the stabilizer 10 may have a sharpened edge to facilitate insertion of the stabilizer 10 in bone. For example, in the illustrated embodiment, each projections 20A-F includes a radially distal edge 28A-F that tapers to form a sharpened edge.

The stabilizer 10 may be constructed of a biocompatible material such as, for example, a metal, such as stainless steel or titanium, a ceramic, a polymer, or a composite thereof.

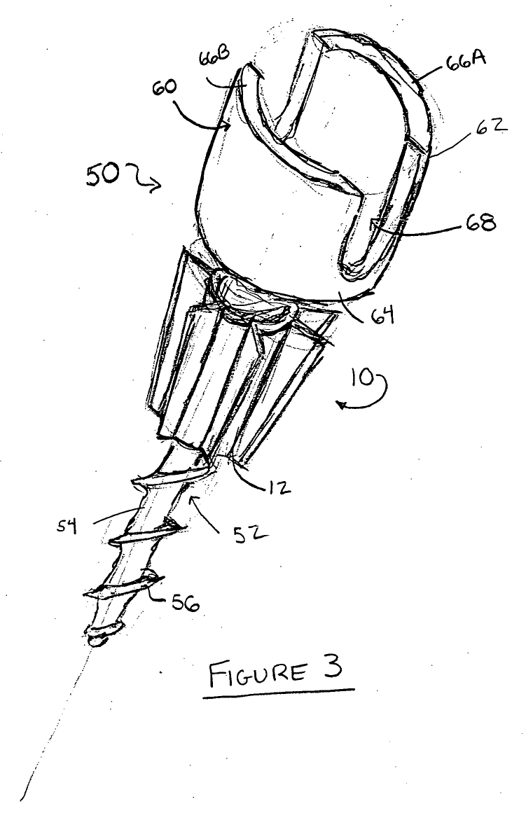

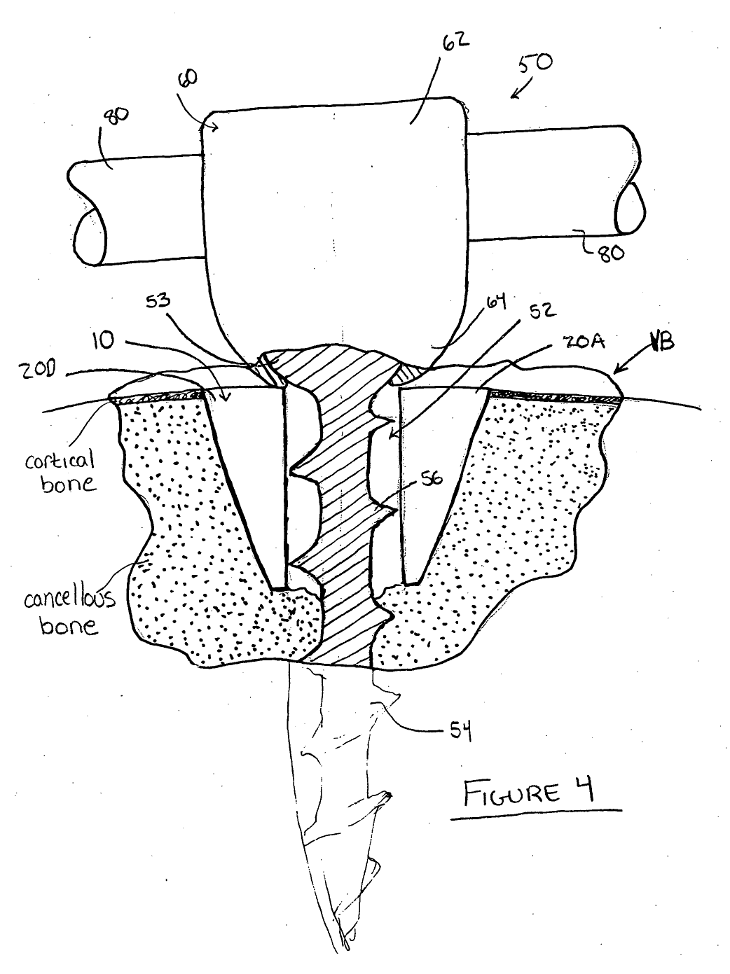

FIGS. 3 and 4 illustrate the exemplary stabilizer 10 coupled to an exemplary bone anchor assembly 50. The exemplary bone anchor assembly 50 includes a bone screw 52 having a proximal head 53 and a distal shaft 54. At least a portion of the shaft 54 of the bone screw 52 includes bone engaging threads 56. The exemplary bone anchor assembly 50 includes a receiver member 60 having a proximal portion 62 for receiving a spinal fixation element, such as, for example, a spinal rod 80, and a distal portion 64 for receiving the proximal head 53 of the bone screw 52. In the exemplary embodiment, the proximal portion 62 of the receiver member 60 is generally U-shaped and includes a pair of spaced apart legs 66A, 66B that define a groove 68 for receiving the spinal fixation element. In the exemplary embodiment, the bone anchor assembly 50 is a polyaxial, e.g., the bone screw 52 is adjustable to a plurality of orientations relative to the receiver member 60. Examples of polyaxial bone anchor assemblies are described in detail in U.S. Pat. No. 5,207,678, U.S. Patent Application Publication No. 2002/0058942, U.S. Patent Application Publication No. 2003/0100896, U.S. Patent Application Publication No. 2004/0181224, and U.S. Patent Application Publication No. 2004/0186473, each of which is incorporated herein by reference. In alternative embodiments, the bone anchor assembly may be monoaxial, e.g., the bone screw 52 may be fixed to the receiver member 60, or may be selectively polyaxial, e.g., the bone screw 52 may be adjustable relative to the receiver member 60 along one or more axes and may be fixed in other axes.

The tubular body 12 of the stabilizer 10 may be positioned about the shaft 54 of the bone screw 52 of the bone anchor assembly 50. For example, body 12 of the stabilizer 10 may be positioned about a proximal portion of the shaft 54 of the bone screw 52, as in the illustrated embodiment. In certain exemplary embodiments, the body 12 of the stabilizer 10 may be rotatable about the axis of the shaft 54 of the bone screw 52. The stabilizer 10 may be an integral component of the bone anchor assembly 50. For example, the body 12 of the stabilizer 10 may be provided about the shaft 54 of the bone screw 52 prior to insertion of the bone anchor into bone. Alternatively, the stabilizer 10 may be a separate component from the bone anchor assembly 50. For example, the stabilizer 10 may be inserted into bone independent of the bone anchor assembly 50, as described in more detail below.

An exemplary method of inserting a bone screw assembly into bone using a stabilizer, such as the exemplary stabilizer 10 described above, may include creating a hole in a portion of a vertebra VB, by for example drilling a hole in the pedicle or other portion of the vertebra. The stabilizer 10 may be inserted into the hole and a bone screw, such as the bone screw assembly 50 described above, may be positioned in the stabilizer 10. For example, the shaft 54 of the bone screw 52 may be introduced into the body 12 of the stabilizer 10 by rotating the bone screw 52 to cause the external threads 56 on the shaft 54 to engage the internal threads 22 on the inner wall 16 of the body 12 of the stabilizer 10.

Once implanted in the vertebra, the stabilizer 10 operates to distribute the forces applied to the bone anchor assembly 50 over an increased area of bone. Referring to FIG. 4, for example, the stabilizer 10 operates to inhibit rotation of the bone anchor assembly 50 and distribute to bone anchor forces over an increased area of the cortical bone and cancellous bone of the vertebra.

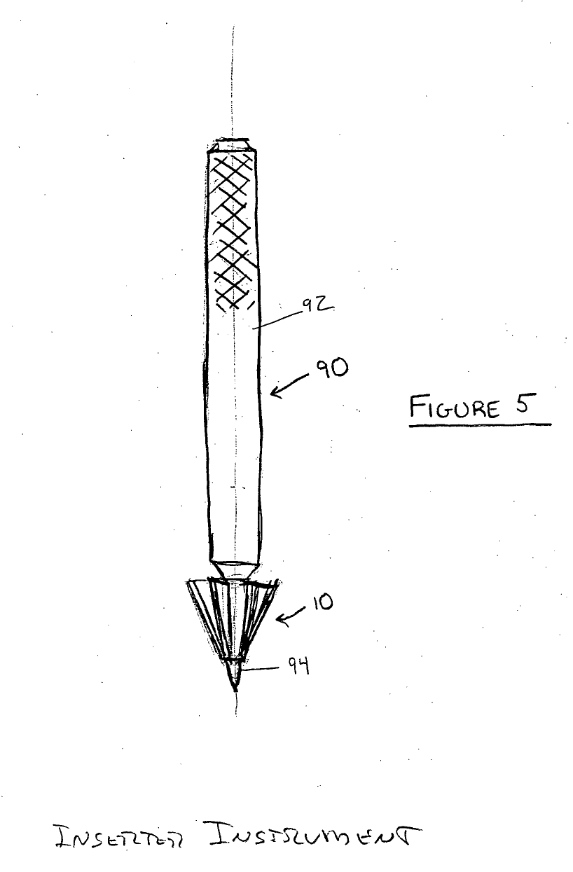

FIG. 5 illustrates an exemplary instrument 90 for positioning a stabilizer, such as the exemplary stabilizer 10 describe above, into bone. The exemplary instrument 90 includes an elongated shaft 92 that terminates at a distal tip 94. The distal tip 94 is sized and shaped to be received within the lumen 18 of the body 12 of the stabilizer 10 and terminates at a pointed tip to facilitate insertion into a hole in bone. In use, the distal tip 94 of the instrument may be inserted into the body of the stabilizer 10 and positioned within a hole formed in the bone. The proximal end of the instrument 90 may be impacted by a mallet or other suitable instrument to drive the distal tip 94 of the instrument 90 and the stabilizer into the hole in the bone.

While the devices and methods of the present invention have been particularly shown and described with reference to the exemplary embodiments thereof, those of ordinary skill in the art will understand that various changes may be made in the form and details herein without departing from the spirit and scope of the present invention. Those of ordinary skill in the art will recognize or be able to ascertain many equivalents to the exemplary embodiments described specifically herein by using no more than routine experimentation. Such equivalents are intended to be encompassed by the scope of the present invention and the appended claims.

1. A stabilizer for a bone anchor, the stabilizer comprising:
a tubular body having an outer wall and an inner wall, the inner wall defining a lumen sized and shaped to receive a portion of the bone anchor, and
a plurality of projections spaced about and extending from the outer wall of the tubular body, the projections being configured to engage bone and distribute forces on the bone anchor to the bone.

2. The stabilizer of claim 1, wherein at least one of the projections tapers from a proximal end of the tubular body to a distal end of the tubular body.

3. The stabilizer of claim 1, wherein at least one of the projections includes a sharpened edge to facilitate insertion of the bone anchor into bone.

4. The stabilizer of claim 1, wherein the inner wall of the tubular body includes internal threads to facilitate receipt of the portion of the bone anchor.

5. The stabilizer of claim 1, wherein the projections are spaced equally about the outer wall of the tubular body.

6. The stabilizer of claim 1, wherein the projections are similarly sized and shaped.

7. The stabilizer of claim 1, wherein the tubular body and the projections are constructed from a biocompatible material.

8. A spinal bone anchor assembly comprising:
a bone screw having a proximal head and a distal shaft, at least a portion of the distal shaft including bone engaging threads,
a receiver member having a proximal portion for receiving a spinal fixation element and a distal portion for receiving the proximal head of the bone screw, and
a stabilizer having a tubular body positioned about a portion of the shaft of the bone screw and a plurality of projections spaced about and extending from the tubular body, the projections being configured to engage bone to inhibit rotation of the stabilizer and distribute forces on the bone anchor to the bone.

9. The bone anchor assembly of claim 8, wherein the bone screw is adjustable to a plurality of orientations relative to the receiver member.

10. The bone anchor assembly of claim 8, wherein the bone screw is fixed to the receiver member.

11. The bone anchor assembly of claim 8, wherein the stabilizer is rotatable about the shaft of the bone screw.

12. The bone anchor assembly of claim 8, wherein the shaft has a proximal non-thread section and a distal threaded section and the stabilizer is positioned about the proximal non-threaded section of the shaft.

13. The bone anchor assembly of claim 8, wherein at least one of the projections tapers from a proximal end of the tubular body to a distal end of the tubular body.

14. The bone anchor assembly of claim 8, wherein at least one of the projections includes a sharpened edge to facilitate insertion of the shaft of the bone screw into bone.

15. The bone anchor assembly of claim 8, wherein the tubular body includes internal threads to facilitate receipt of the shaft of the bone screw.

16. The stabilizer of claim 1, wherein the projections are spaced equally about the outer wall of the tubular body.

17. A method of inserting a bone screw into a vertebra, the method comprising:
creating a hole in a portion of the vertebra,
inserting a stabilizer in the hole, the stabilizer having a tubular body and a plurality of projections spaced about and extending from the tubular body, and
positioning the shaft of a bone screw in the stabilizer.

18. The method of claim 17, wherein positioning the shaft of the bone screw in the stabilizer includes rotating the shaft to engage external threads on the shaft with internal threads within the tubular body.

19. The method of claim 17, wherein creating the hole in a portion of the vertebra comprises drilling a hole in a pedicle of the vertebra.

20. The method of claim 17, wherein inserting the stabilizer comprises impacting the stabilizer with an instrument to drive the stabilizer in the hole.
